(19) United States
(12) Patent Application Publication
(10) Pub. No.: US 2024/0299020 A1
(43) Pub. Date: Sep. 12, 2024

(54) ADAPTER FOR SURGICAL RETRACTOR POSITIONING DEVICE

(71) Applicant: The Feinstein Institutes for Medical Research, Manhasset, NY (US)

(72) Inventors: Jonathan Robert Danoff, Great Neck, NY (US); Todd Goldstein, Albertson, NY (US)

(21) Appl. No.: 18/583,007

(22) Filed: Feb. 21, 2024

Related U.S. Application Data
(60) Provisional application No. 63/450,287, filed on Mar. 6, 2023.

(51) Int. Cl. A61B 17/02

(52) U.S. Cl. CPC A61B 17/02; A61B 2017/0287

(57) ABSTRACT

An adapter connects a surgical instrument, such as a retractor, with a self-retaining retraction device. The adapter includes a retractor interface sized and shaped to releasably connect with the proximal end of a surgical retractor. The adapter also includes a device interface sized and shaped to releasably connect with the self-retaining device. The adapter allows the same self-retaining device to be used with different retractors. The adapter may also include a joining portion that holds the longitudinal axis of the retractor at a selected angle with respect to the self-retaining device.

### RELATED APPLICATIONS

This application claims priority under 35 U.S.C. § 119 to U.S. Provisional Patent Application No. 63/450,287, filed on Mar. 6, 2023. The disclosure of that application is incorporated herein by reference.

### BACKGROUND

### Field

This disclosure relates to an adapter for connecting a surgical instrument to a positioning device for holding the instrument relative to a surgical incision and for applying tension to the instrument. More particularly, the disclosure relates to an adapter that allows a variety of medical instruments to be connected with a selected positioning device and that provides a select angular orientation between the surgical instrument and the positioning device.

Some medical procedures require that force be applied to hold tissues away from a portion of the body being treated or visualized during surgery. Retractors and clamps of various kinds are commonly used in surgical procedures wherein the retractor or clamp is positioned within the cavity of a surgery patient so as to position or locate a member or organ of the body of the patient at a desired location to enable a surgeon to obtain free access to a portion of the body or body cavity during a surgical procedure.

Medical personnel can apply force using a retractor in any selected direction. Such personnel can change their grip on the retractor to gain mechanical advantage along the selected direction. For example, a surgical assistant may rotate their wrist, or grip a retractor using different gripping surfaces to comfortably apply force in a desired direction.

Typically, surgical assistants insert a retractor or retractors into an opening surgically created in the body of a patient. The assistant then holds the retractor(s) in place while the surgeon carries out the surgical procedure. The need for such surgical assistance increases the cost of the surgical procedure and the presence of the assistant in the operating field requires the surgeon to avoid the hands and arms of the assistant or assistants that are holding the retractor in position during the surgical procedure. In addition, for procedures where tissues need to be retracted for long periods of time, for example, total hip replacements, applying retraction may lead to muscle fatigue. Further, not all instruments are designed to provide a comfortable grip. This can lead to hand pain and fatigue.

Moreover, procedures using robotic or computer navigation technology may require line of sight cameras or tracking systems to visualize various landmarks and/or markers around the operative field. In these cases, a surgical assistant applying force on the retractor must move out of the way to avoid blocking the visualization of these landmarks. This may be inconvenient. Thus, there is a need for a system to facilitate holding retraction during surgery that does not require an assistant to physically hold and apply force to the retractor during a surgical procedure.

Numerous devices and systems which allow retractors to be positioned and that hold their position and the force applied indefinitely are available from various manufacturers. One such device is the Gripper™, from MedEnvision of Aarschot, Belgium (https://medenvision.com/gripper/). The Gripper™ is a self-retaining retraction device that is fixed to an anchor point, for example, a post connected with the surgical table and that connects with a retractor or other medical instrument to retract tissue in a selected direction and with a selected force. Once the retractor is placed and connected with the device, the retractor is held until released by medical personnel.

Use of such a self-retaining device avoids some of the problems associated with having medical personnel apply retraction. Self-retaining retraction devices, however, suffer from a number of problems. Such devices generally have a connection portion shaped to receive insertion of the handle of a single commercially available surgical retractor. The connection portion must be designed to securely hold the retractor handle with a particular shape and size. The handle portions of retractors made by different manufacturers may have different dimensions or shapes. To accommodate different retractors, multiple self-retaining retraction devices may be required, each including a shape specific to hold a specific retractor handle design.

For some surgical procedures, linear force is applied to the retractor to pull tissue directly away from a surgical wound. In other procedures, instead or, or in addition to linear force, torque must be applied to the retractor. For example, during total joint replacement procedures, layers of musculature around the joint may need to be moved and held out of the way for long periods of time. This muscular tissue is relatively stiff, requiring significant force to be displaced. A Hohmann retractor may be used in such procedures. The Hohmann retractor has a spike at its tip designed to engage with bone, for example, on the patient's femur. A widened portion of the retractor near this spiked tip contacts the tissue to be retracted. The spike forms a fulcrum so that force applied to the handle of the Hohmann retractor creates torque on the instrument. Mechanical advantage provided by the relatively long distance from the tip to the handle in relation to the short distance between the tip and the widened portion allows a medical practitioner to apply retraction to musculature around the joint being replaced. In order to apply such torque efficiently, the angle between the axis of the retractor and the applied force may be selected to be as close to 90 degrees as possible. Where such torque is applied by medical personnel, there is no difficulty in adjusting the angle between the retractor and the applied force. Where a known self-retaining retraction device is used, however, it may be inconvenient to adjust the angle of the applied force to maximize torque. In addition, self-retaining retraction devices require that a cord extend from the device to a fixed point. Depending on the position of the retractor, these cords may be at sharp angles relative to the retractor, which may make the device more difficult to use or require the cord to stretch across a part of the surgical field. In addition, applying force to a retractor using known retraction devices may increase stress on the bone or tissue being displaced. There is a need for a device that can adjust the angle between a retractor and a self-retaining retraction device to decrease the stress on the bone and/or tissues being tensioned by orienting the direction of the applied force to decrease the risk of bone fracture, nerve traction, or other compression injury to the patient's tissue.

### SUMMARY

The present disclosure relates to an adapter for a self-retaining retraction device that addresses these and other difficulties.

According to one aspect of the disclosure, an adapter is formed with a retractor interface at a distal end and a self-retaining positioning device interface at a proximal end. The retractor interface may be shaped to receive a plurality of different retractors and to securely connect the retractor to the adapter. The adapter connects with the positioning device using a standard connection arrangement. According to one embodiment, a kit of adapters may be provided along with a known self-retaining retractor positioning device. Such a self-retaining positioning device includes the mechanical retracting systems disclosed in U.S. Pat. No. 9,949,729, issued Apr. 24, 2018, the entirety of which is incorporated herein by reference. The kit includes adapters shaped to connect with a plurality of different retractors. In use, a medical practitioner selects the appropriate adapter to connect with the retractor that will be used during a surgical procedure and connects the retractor with the positioning device using the adapter.

According to one embodiment, the retractor or instrument interface includes a fixing device for securely connecting the retractor with the adapter. The fixing device may form an interference fit between the retractor handle and the adapter. According to one embodiment, the retractor interface is shaped to receive a standard retractor handle, such as a rectangular handle with a thickness of between about 2.0 millimeters (mm) and about 4.5 mm and a width between about 25 mm and about 33 mm. According to another embodiment, the fixing device includes a key and keyway designed to engage with one or more holes through the handle of the retractor.

According to another embodiment, openings are provided through the walls of the retractor interface and similar sized openings are provided through the retractor handle. These openings form a keyway. A removable pin or key shaped to fit through the keyway is also provided. Insertion of the key into the keyway fixes the adapter with the retractor. According to one embodiment, the key forms an interference fit with the keyway. Removal of the key allows the retractor to be separated from the adapter. According to a further embodiment, multiple keys and keyways may be provided. According to a still further embodiment, multiple keys are connected with a base and positioned so when the base is moved toward the adapter, the keys are each inserted simultaneously into the keyways.

According to other embodiments, keys and keyways are provided with locking mechanisms that allow medical personnel to secure the adapter to the instrument and removable fix the key in the keyway. Such locking devices may include a tab that rotatably engages with a slot on the adapter so that rotation of the key engages the tab with the slot to hold the key in the keyway. According to another embodiment, the key includes a threaded connector and the keyway includes a threaded opening to removably engage with the threaded connector.

According to a further embodiment, a joining portion connects the retractor interface and the positioning device interface. The retractor interface defines a retractor longitudinal axis and the positioning device interface defines a device longitudinal axis. According to one embodiment, the joining portion holds the retractor interface and the device interface so that the retractor axis and the device axis are co-linear, that is, there is a 180 degree angle between the device axis and the retractor axis. According to another embodiment, the joining portion holds the device axis and the retractor axis at a specified angle in the sagittal or coronal plane. According to one embodiment, the specified angle is between about 0 degrees and about 180 degrees. According to one preferred embodiment, the specified angle is about 135 degrees. According to another preferred embodiment, the angle is about 60.5 degrees. According to one embodiment, the angle is selected to optimize the direction of pull of the device to properly tension the tissues, allow for self-retraction, and to minimize stress/trauma to the surrounding tissues, bone, nerves.

According to a further embodiment, the joining portion holds the retractor longitudinal axis and the device longitudinal axis at angular orientations with respect to one another along two different planes to selectively control the force vector and line of pull of the retractor to adjust tension on the body part or to better visualize an area of interest.

According to one embodiment, an adapter according to the disclosure is designed to connect with a known self-retaining retraction device, such as a Gripper™ device, described in U.S. Pat. No. 9,949,729, issued Apr. 24, 2018 and incorporated herein by reference. Such a device may be designed to accept insertion of the handle of a known retractor, such as a Hohmann retractor. According to one embodiment, the device interface of the adapter includes a tongue with the same size and shape as a known retractor handle. According to one embodiment, the tongue has a rectangular cross section with a thickness of about 2 mm and a width of about 30 mm. Insertion of the tongue into a known self-retracting device releasably fixes the adapter with the device. The tongue may form an interference fit with the gripper device. The gripper device may also include a locking mechanism or screw that can be actuated to removably fix the tongue of the adapter with the device.

According to a further embodiment, the device interface includes one or more walls shaped to contact outer side surfaces of retraction device. According to one embodiment, the walls improve the mechanical connection of the adapter to the retraction device. According to another embodiment, one or more of the walls include a lip along a top edge that is positioned to contact a top outer surface of the retraction device to further improve the mechanical connection of the adapter with the retraction device.

According to a further embodiment, the device interface connects with the handle of a surgical instrument, such as a retractor to provide a comfortable gripping surface.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1A is a perspective view of a self-retaining retraction device connected with an adapter according to an embodiment of the present disclosure;

FIG. 1B is a perspective view showing the assembly of an adapter according to embodiments of the present invention onto a self-retaining retraction device;

FIG. 2 is a perspective view of an adapter according to an embodiment of the disclosure;

FIG. 3 is a perspective view of an exemplary self-retaining retraction device that can be used with the adapter of FIG. 2;

FIGS. 4A, 4B, 4C, 4D, and 4E are exemplary surgical retractors that can be used with the adapter of FIG. 2;

FIG. 5A is an elevation view of an adapter according to an embodiment of the disclosure, FIG. 5B is a perspective view of a key used to connect a surgical retractor to the adapter of FIG. 5A, and FIG. 5C shows the adapter of FIG. 5A connected with a surgical retractor and positioned for use in a surgical procedure;

FIGS. 6A to 6C and 6E show views of an adapter according to another embodiment of the disclosure and FIG. 6D is a perspective view of a key used to connect a surgical retractor to the adapter of FIGS. 6A to 6C and 6E;

FIGS. 6F to 6H show views of an adapter according to the embodiment of FIGS. 6A to 6C and 6E connected with a retractor and with a self-retaining retraction device;

FIGS. 7A to 7D show views of an adapter according to a further embodiment of the disclosure;

FIG. 8A shows an adapter according to the embodiment of FIGS. 7A to 7D connected with a self-retaining retraction device and FIG. 8B shows the adapter and retraction device FIG. 8A connected with a surgical retractor and positioned for use in a surgical procedure;

FIG. 9 shows an adapter according to a further embodiment of the disclosure;

FIGS. 10A and 10B show an adapter according to a further embodiment of the disclosure;

FIGS. 11A and 11B show an adapter according to a further embodiment of the disclosure;

FIG. 12 shows a key used with the adapter of FIGS. 11A and 11B;

FIG. 13 shows an adapter according to a further embodiment of the disclosure;

FIG. 14 shows an adapter according to a further embodiment of the disclosure;

FIGS. 15A and 15B show a key used with the adapter of FIG. 13 or FIG. 14;

FIG. 16 shows an adapter according to a further embodiment of the disclosure;

FIG. 17 shows a key used with the adapter of FIG. 16;

FIGS. 18A and 18B show steps for securing the key of FIG. 17 with the adapter of FIG. 16; and

FIG. 19 shows a key according to a further embodiment of the disclosure

### DETAILED DESCRIPTION

For purposes of this disclosure, the terms “distal,” “distally,” “distal of” and the like will be used throughout this disclosure to refer to the direction or relative position toward the body of a patient being treated using the device. The terms “proximal,” “proximally,” “proximal of” and the like will be used throughout this disclosure to refer to the direction away from the body of a patient being treated using the device.

Embodiments are described in terms of treatment of a human patient. The disclosure is not limited to devices to treat humans and is applicable to perform veterinary procedures on animals.

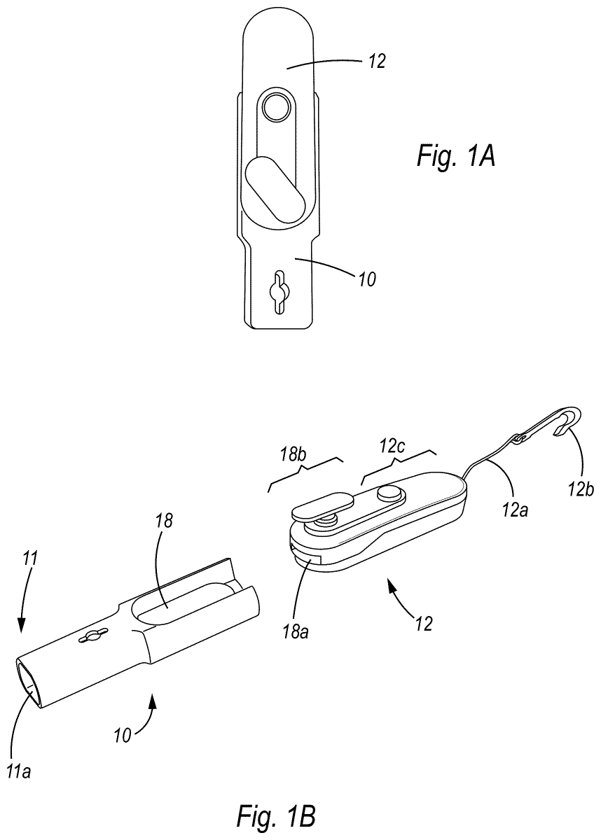

FIG. 1A shows an adapter 10 according to embodiments of the disclosure connected with a self-retaining retraction device 12. According to one embodiment, device 12 is a mechanical retracting system disclosed in U.S. Pat. No. 9,949,729, issued Apr. 24, 2018 (hereinafter “the '729 patent”), the entirety of which is incorporated herein by reference. FIG. 1B shows adapter 10 aligned for connection with device 12. Device 12 has an opening 18a at the distal end 18b of the device 12. Opening 18a is shaped to accept insertion of the proximal end of a surgical retractor into an attachment mechanism 18b within device 12 to securely engage the retractor. According to one embodiment, opening 18a is sized and shaped to connect with the handle of a standard Hohmann surgical retractor or a similar shaped flat proximal end instrument. According to a further embodiment, opening 18a accepts insertion of a retractor handle with a thickness of between about 2 mm and about 4.5 mm and a width between about 25 mm and 35 mm. Embodiments described below connect surgical instruments with the mechanical retraction system disclosed in the '729 patent, but adapters that connect retractors and other surgical instruments with other self-retaining retraction devices 12 are also within the scope of the present disclosure.

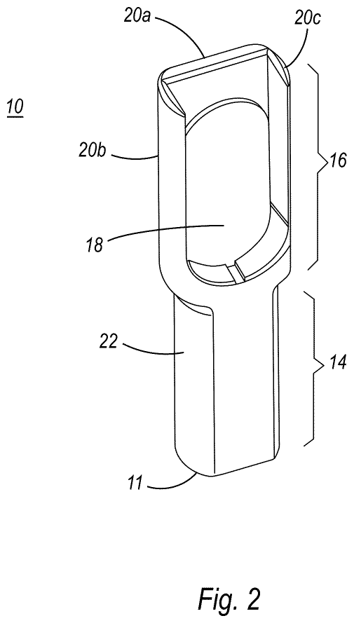

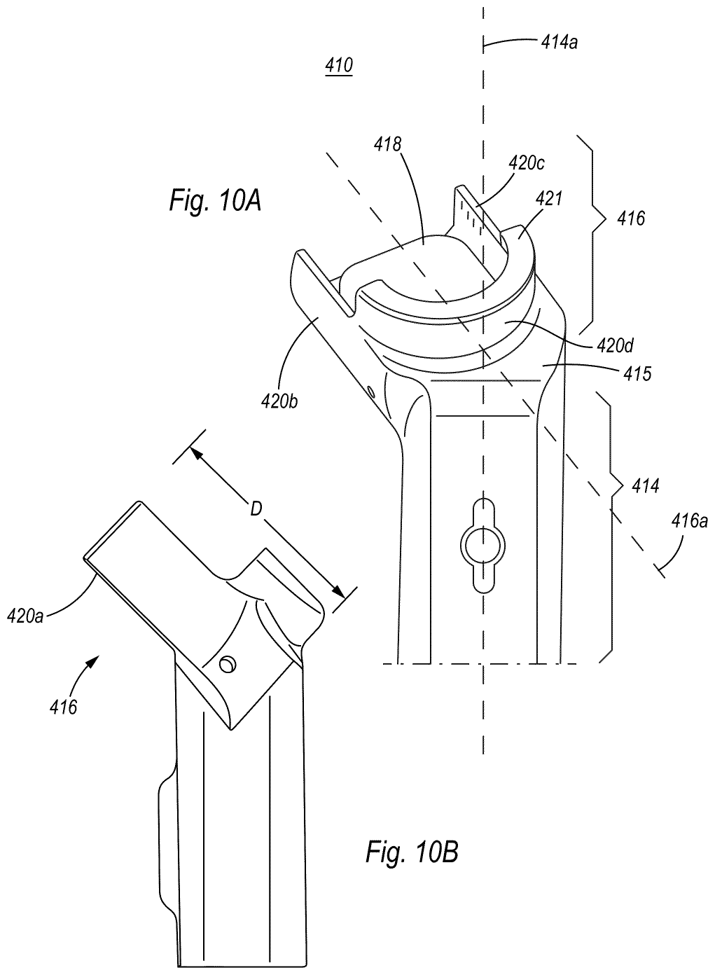

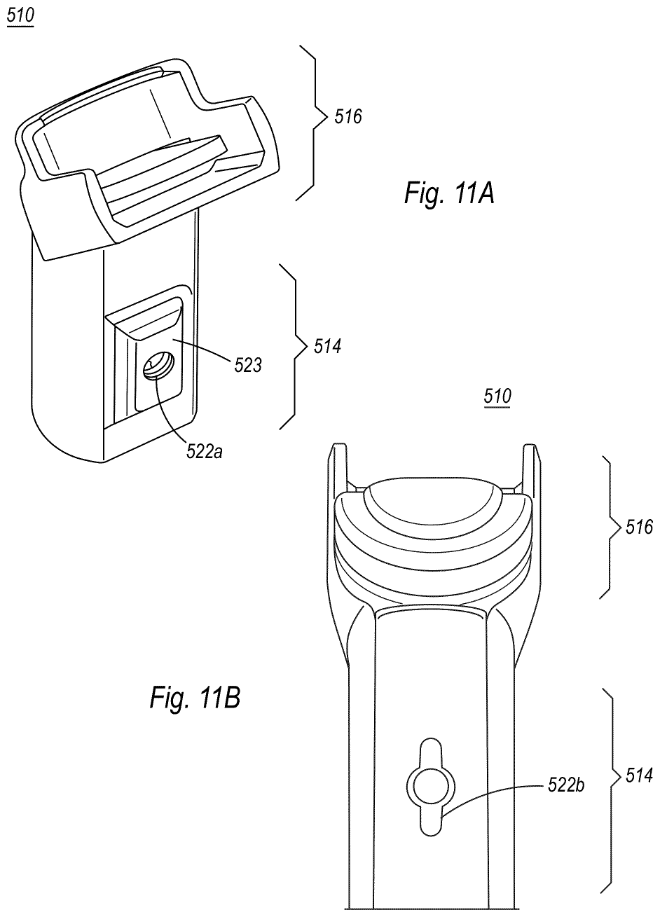

As shown in FIG. 2, according to one embodiment adapter 10 has an instrument interface 14 at its distal end and a self-retaining retraction device interface 16 at its proximal end. Device interface 16 includes tongue 18 with dimensions suitable for being inserted into opening 18a and for being securely retained by attachment mechanism 18b to fix adapter 10 onto device 12. An instrument opening 11 is provided at the distal end of instrument interface 14 into an internal cavity 11a. Opening 11 and cavity 11a are sized and shaped to accept insertion of the proximal end of a surgical retractor or other medical instrument and to securely hold the retractor or instrument. According to embodiments of the disclosure, opening 11 has a shape different from the shape of opening 18a. That is, adapter 10 allows a different medical instrument to be connected with device 12 than the instrument that device 12 was designed for. According to one embodiment of the disclosure, a kit of adapters 10 is provided to a medical practitioner, where each adapter has the same shaped tongue 18 to connect with opening 18a of device 12 and different openings 11 to connect with different instruments. In the embodiment of FIG. 2, tongue 18 is surrounded on two sides by base 20a and walls 20b and 20c. The space between tongue 18 and base and walls 20a, 20b, 20c is selected so that the base and walls engage with outer side surfaces of the distal end 18b of device 12 to provide a mechanically secure engagement between device 12 and adapter 10. According to other embodiments shown in FIGS. 10a-11C, a lip 421 may be provided along the tops of one or more of the walls 20b, 20c to contact a top surface of device 12.

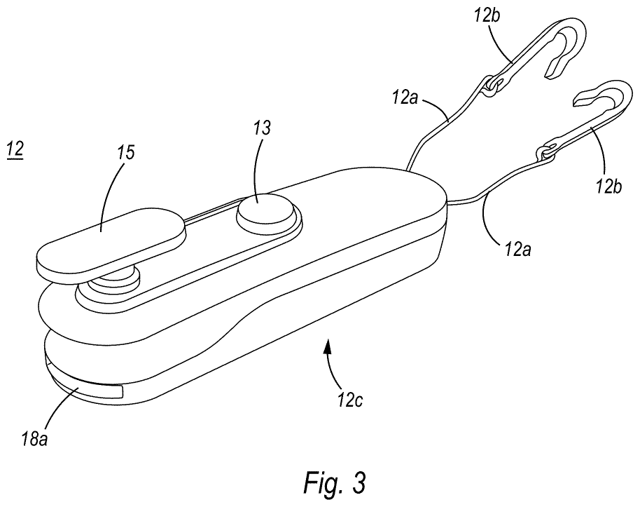

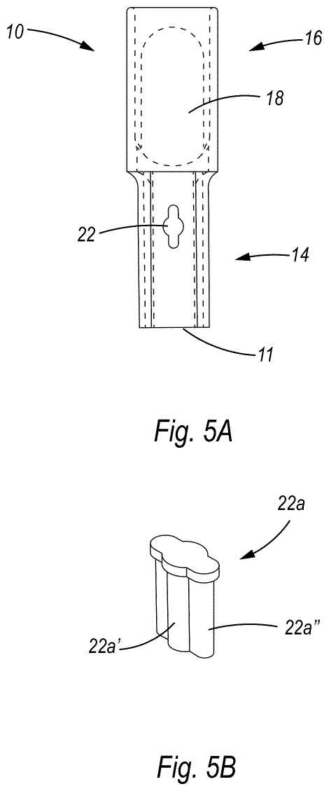

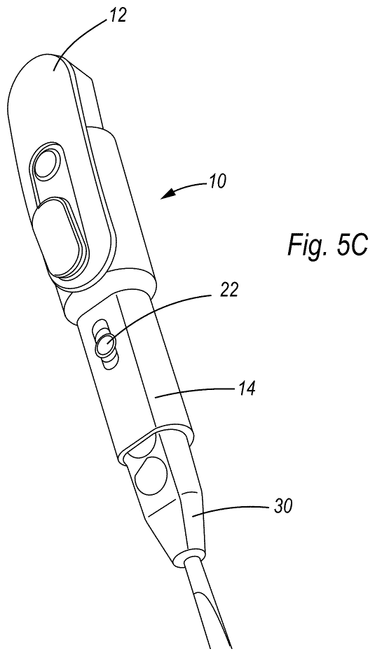

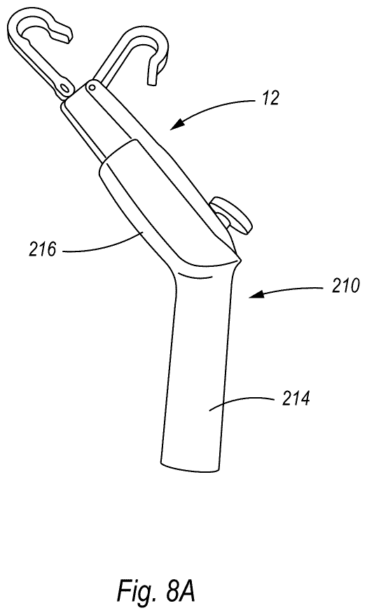

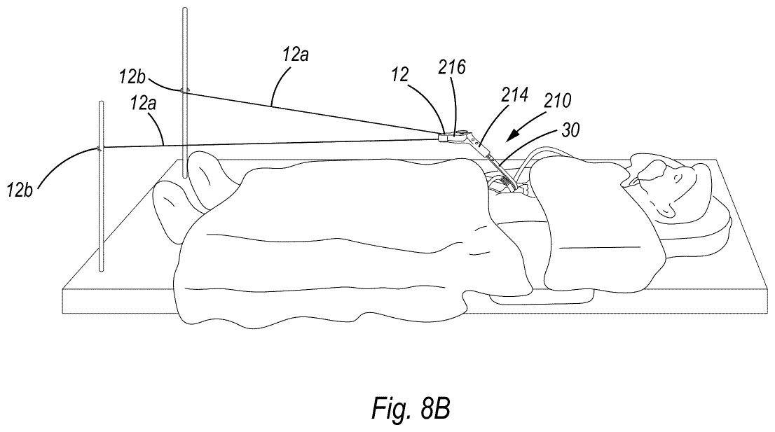

FIG. 3 shows a device 12 according to embodiments of the disclosure. Device 12 includes one or more wires or cords 12a that extend out from the proximal end of the device. A hook, loop, or other connection device 12b is fixed to the proximal end of the cord. A self-retaining retraction mechanism 12c is provided within device 12. Release button 13 allows the surgeon or other medical practitioner to set the position of the adapter. According to one embodiment, an operator depresses the release button 13 to release tension on cords 12a, allowing them to be pulled out from the device. One or more connection devices 12b at the ends of the cords are connected with anchor points outside the surgical field, such as posts connected with the surgical table. Device 12 is connected with adapter 10 and a retractor 30, shown for example, in FIG. 5C. With release button 13 depressed, the retractor is positioned at the appropriate location and orientation within the surgical field. Because the cords 12a are slack, device 12 does not interfere with the positioning of the retractor. Once the practitioner is satisfied that the retractor is properly positioned, the practitioner releases the release button 13, causing retraction mechanism 12c to pull cords 12a inward. This sets the tension and length of cords 12a and holds the current position of device 12, along with the surgical instrument connected with the adapter, as will be explained below. This tension pulls device 12, as well as a medical instrument connected with device 12, in the direction of the anchor points, or the resultant vector direction when two or more cords 12a and connectors 12b are connected to different anchor points, as shown in FIG. 8B.

Device 12 may include a locking device 15 to securely connect the device with a surgical retractor or other instrument. According to one embodiment, locking device 15 comprises a threaded screw that engages with corresponding threads on the body of device 12. Rotation of locking device 15 relative to the body of device 12 in one direction drives the screw into contact with the handle of the retractor to securely connect the retractor with device 12. Rotation of the locking device 15 in the other direction releases pressure on the retractor handle, allowing the retractor to be separated from device 12. When device 12 is connected with adapter 10, locking device 15 applies pressure to tongue 18a to secure the device to the adapter.

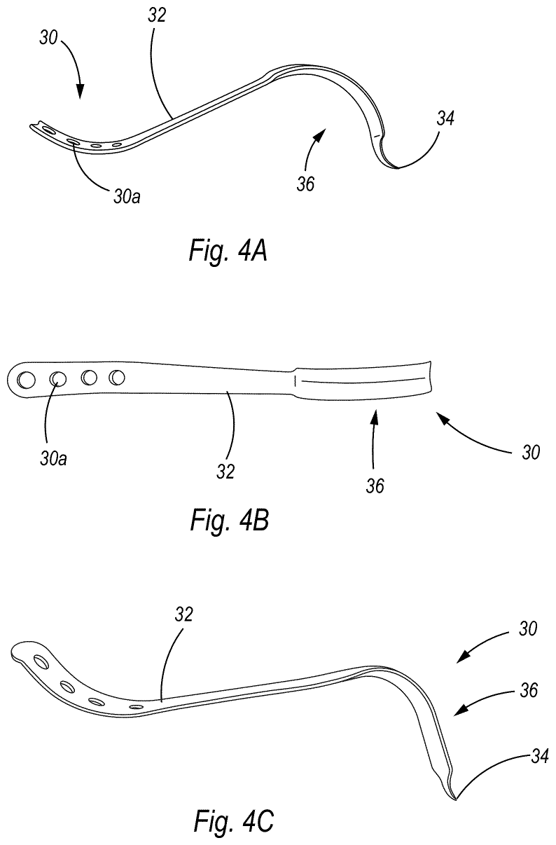

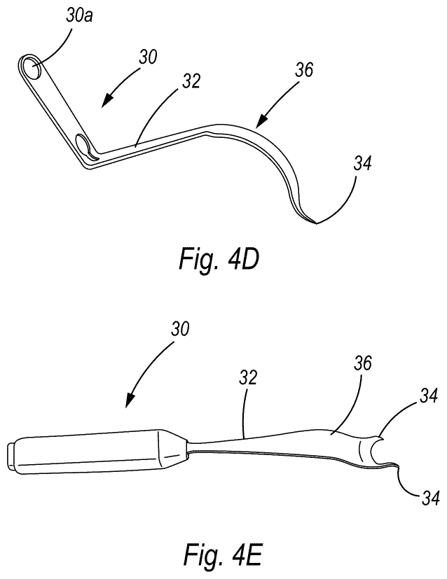

FIGS. 4A to 4C show a surgical retractor 30 that may be used with embodiments of the present disclosure. Openings 30a are provided near the proximal end of the retractor. Openings 30a may be sized to engage with a locking mechanism provided within opening 11 of adapter 10, as shown in FIG. 1B. Insertion of the proximal end of retractor 30 into opening 11 connects the retractor 30 with adapter 10. Body 32 of retractor 30 extends in the distal direction with at least a portion of the retractor arranged along an instrument axis. Tissue engaging surface 36 is provided near the distal end of retractor 30. Force applied to retractor 30 causes surface 36 to engage with a selected portion of tissue within a surgical incision. Force applied to the retractor, for example, by a device 12 connected with adapter 10, moves the selected tissue to facilitate a surgical procedure. According to one embodiment, retractor 30 includes one or more tips 34 that allows the retractor 30 to pivot about a point of contact, for example, where tip 34 contacts bony tissue. FIGS. 4D and 4E show examples of other types of retractors suitable for use with adapter 10 of the disclosure. The disclosure is not limited to use with these instruments and encompasses embodiments for positioning other medical devices.

According to one embodiment, retractor 30 is releasably connected with adapter 10 using openings 30a. FIG. 5A shows adapter 10 according to one embodiment. Opening 22 on adapter 10 is provided through at least one wall of retractor interface portion 14. When the proximal end of retractor 30 is inserted into opening 11 of adapter 10, hole 30a of the retractor aligns with opening 22 to form a keyway. As shown in FIG. 5B, key 22a is shaped to have a cross section to fit into opening 22. According to one embodiment, key 22a is formed by a central portion 22a′ and one or more lobes 22a″. Key 22a is inserted through the keyway formed by openings 22a and 30a to fix retractor 30 with adapter 10. Shapes of opening 22 and key 22a may be selected to connect different types of retractors or other medical instruments 30 with adapter 10. Key 22a and opening 22 may be sized to create an interference fit that holds the key within the opening and secures retractor 30 with adapter 10 until the key is pulled from the opening. FIG. 5C shows adapter 10 connected with retraction device 12 and with the handle of retractor 30. In this embodiment, the retractor is a Biomet Mueller 2 Straight Retractor. According to other embodiments, interface portion 14 is shaped to secure adapter 10 to other medical instruments that are to be positioned using a self-retaining retraction device 12.

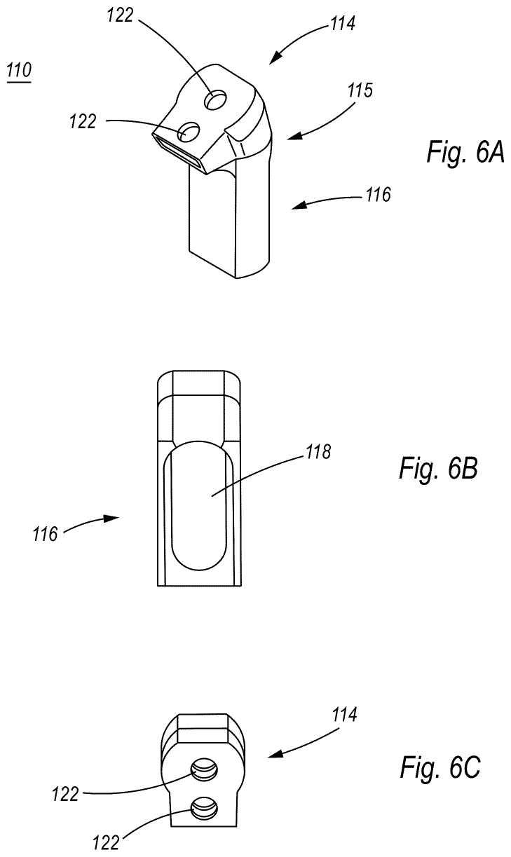

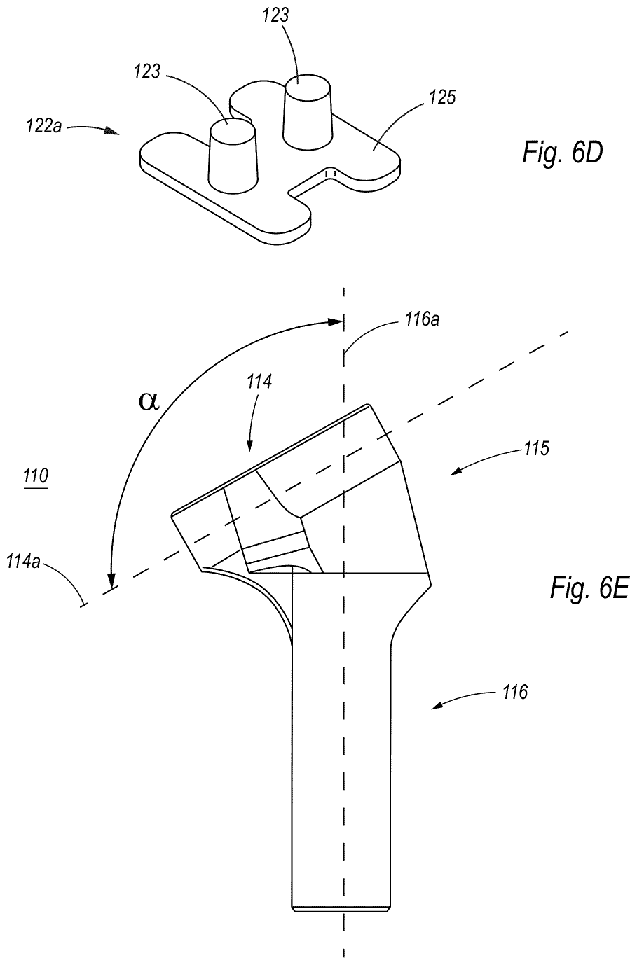

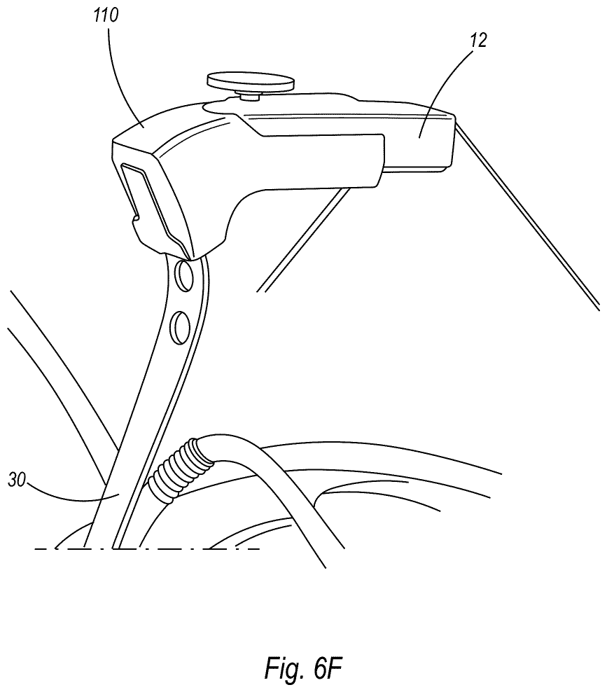

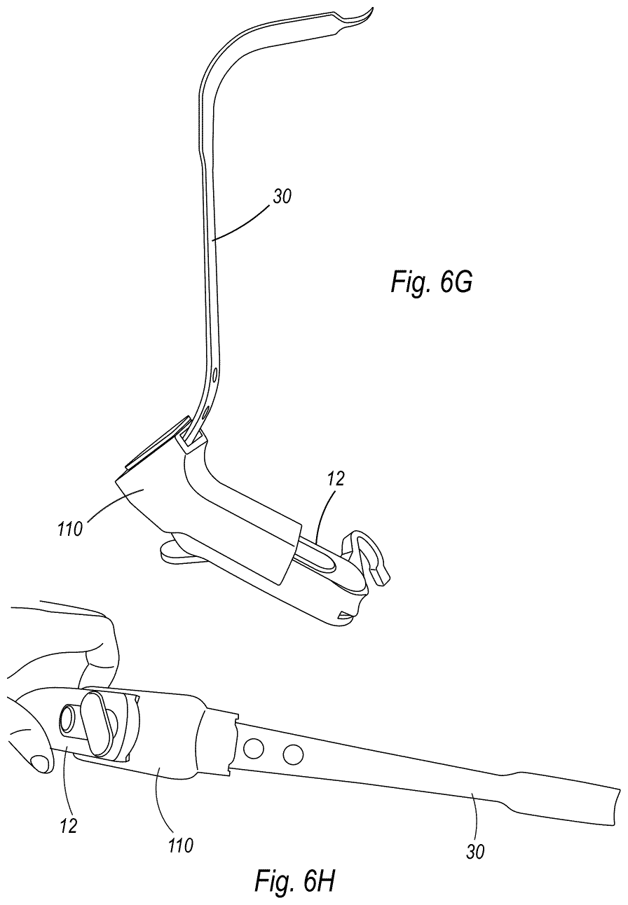

FIGS. 6A through 6H show an adapter 110 according to another embodiment of the disclosure. Device interface 116 is provided at one end of adapter 110 and defines device axis 116a, as shown in FIG. 6E. As can be seen in FIG. 6B, adapter 110 includes tongue 118. As in the previous embodiments, tongue 118 is sized and shaped to be received into opening 18a in device 12 to releasably fix adapter 10 and device 12 together. Retractor interface 114 is provided at the distal end of adapter 110 and defines an instrument axis 114a. Joining portion 115 connects interfaces 114 and 116 with one another. Joining portion 115 holds interfaces 114 and 116 at a selected angle with respect to one another such that device axis 116a and instrument axis 114a are at a selected angular separation α. That is, when retractor 30 is inserted into the retractor interface 114 and a device 12 is connected with device interface 116, a longitudinal axis of the retractor 30 is at the selected angle relative to a longitudinal axis of device 12. The selected angle can be in the sagittal or coronal plane or can be oblique to the sagittal and coronal planes. According to one embodiment, a variety of adapters 10 with various selected angles are provided as a kit that allows a practitioner to select an adapter with the appropriate angle.

As shown in FIGS. 6A and 6C, openings 122 are provided through one or more walls of retractor interface 114. According to one embodiment, two openings 122 are provided. Openings 122 may be positioned to align with openings 30a of retractor 30, shown in FIG. 4B. When openings 122 and 30a are aligned they form a keyway. FIG. 6D shows key 122a with two protrusions 123 extending from a base 125. Protrusions 123 are shaped to fit into the keyways to engage the retractor 30 with the adapter 10. Two openings 122 and two protrusions 123 are shown in this embodiment, however, a greater or fewer number of openings and protrusions may be provided with the scope of the disclosure.

In use, a surgeon or other medical professional selects an adapter with a selected angle that is required for the procedure. FIG. 6F shows adapter 110 connected with device 12 and with retractor 30 in use in a surgical field. FIGS. 6G and 6H show a side view and a top view, respectively, of adapter 110 connected with retractor 30 and with device 12. In this embodiment, retractor 30 is a Stryker Hohmann Retractor.

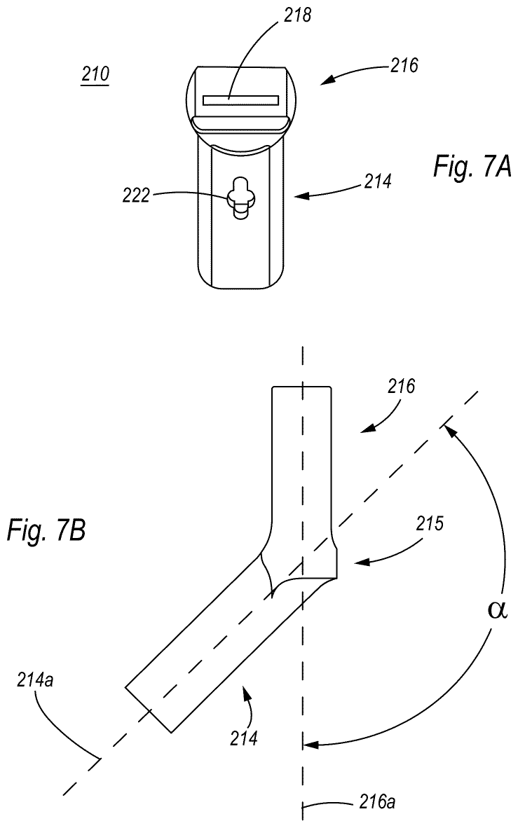

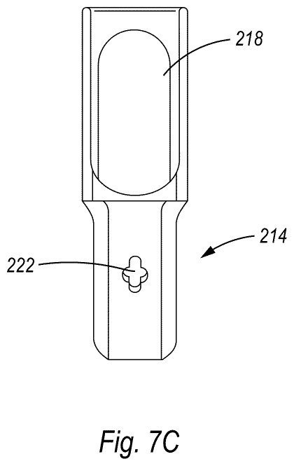

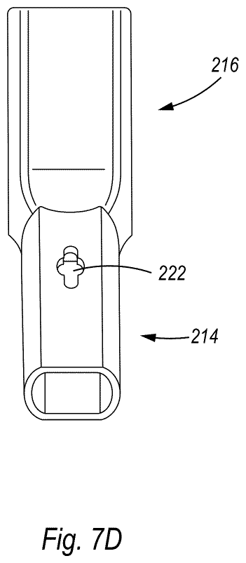

FIGS. 7A through 7D show adapter 210 according to another embodiment of the disclosure. FIG. 8A shows adapter 210 connected with device 12. As with previous embodiments, adapter 210 is formed by device interface 216 connected by joining portion 215 with retractor interface 214. As with previous embodiments, retractor interface 214 provides an opening to receive the proximal end of a surgical retractor or other instrument. According to one embodiment, insertion of retractor 30 into retractor interface 214 forms an interference fit that secures the retractor with the adapter 210. According to a further embodiment, adapter 210 includes one or more openings 222 on the interface 214. Opening 222 aligns with corresponding openings 30a in retractor 30 to create a keyway. If the surgeon wishes to further secure the retractor 30 with adapter 210, a key, such as the key shown in FIG. 5B, is inserted into the keyway. Tongue 218 on device interface 216 is shaped to engage with device 12, as discussed in regard to the previous embodiments. As shown in FIG. 7B, retractor interface 214 defines a retractor longitudinal axis 241a and device interface 216 defines a device longitudinal axis 216a. Joining portion 215 holds interfaces 214, 216 so that axes 214a and 216a are at a selected angle α with one another. Angle α may be from about 0 degrees to about 180 degrees. According to the embodiment shown in FIG. 7B, angle α is about 135 degrees. In the embodiment discussed with regard to FIG. 6A through 6D, the angle α is about 45 degrees. Angle α may be selected to direct the tensile force applied by device 12 onto retractor 30 in a selected direction and to allow wire 12a of device 12 to be located at a convenient position with respect to the surgical field. According to some embodiments, angle α is selected to direct the line of pull along a perpendicular axis as well at various angles.

FIG. 8B shows adapter 210 connected with retraction device 12 and with the handle of retractor 30. Retractor 30 is positioned within a surgical incision to position tissues to facilitate a surgical procedure. Wires 12a connect device 12 with anchor points, such as posts extending from a surgical table. Tension applied by wires 12a on device 12 are communicated through adapter 210 connected with instrument 30 at a selected angle.

According to a further embodiment, an adapter according to the disclosure is used without a self-retaining retraction device. Adapter 10 may be shaped to provide an ergonomically improved grip for a retractor 30. According to one exemplary embodiment, adapter 10 is shaped to increase the girth of the retractor handle making it easier to grip and decreasing stresses on the joints of the medical practitioner's hand while applying force manually. According to another embodiment, adapter 10 is shaped to blunt sharp edges of the retractor handle, making use of the retractor more comfortable.

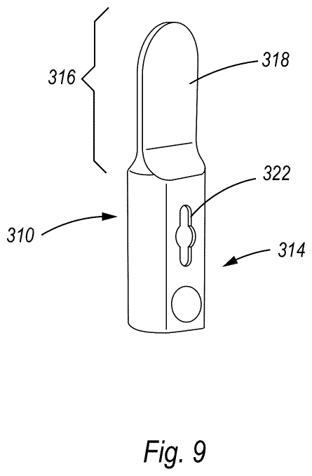

FIG. 9 shows an adapter 310 according to another embodiment of the disclosure. Device interface portion 316 includes tongue 318. Tongue 318 is sized and shaped to engage with opening 18a of device 12, as with the previous embodiments. In this embodiment, tongue 318 is not surrounded by base and walls 20a, 20b, 20c in FIG. 2. In this embodiment, the angle α between the longitudinal axis of interface portion (which is the same as the longitudinal axis of device 12) and the longitudinal axis of the handle of a retractor engaged with adapter 310 is 180 degrees. Retractor interface 314 includes hole 322 that is sized and shaped to accept a key, such as key 22a in FIG. 5B.

FIG. 10A-10C show an adapter 410 according to another embodiment of the disclosure. Instrument interface 414 is connected with device interface 416 by joining portion 415 with instrument axis 414a held at an angle with respect to device axis 416a. According to this embodiment, axes 414a and 416a are at selected angles to one another along two geometric planes.

Device interface 416 includes base 420a, side walls 420b, 420c and end wall 420d. According to one embodiment, end wall 420d includes lip 421. The height of wall 420d and lip 421 are selected so that lip 421 contacts a top outer surface of device 12 to securely fix adapter 410 with device 12. As discussed for the previous embodiments, device interface 416 includes tongue 18 that is shaped to fit into opening 18a of device 12.

As shown in FIG. 10B, device interface 416 defines a depth D between the proximal face of end wall 420d and the proximal ends of side walls 420b and 420c. Distance D is selected to provide a secure connection between device 12 and adapter 410. By providing lip 421 to improve the connection between the adapter and the device, distance D can be minimized to reduce the size and weight of adapter 410. For example, distance D of adapter 410 may be shorter than the corresponding distance of the embodiment shown in FIG. 2, to create a smaller, lighter adapter.

FIGS. 11A to 11C show an adapter 510 according to another embodiment of the disclosure. Device interface 516 connects adapter 510 with device 12, in a similar manner to the embodiments discussed above. Instrument interface 514 is provided at a distal of device interface 516. As shown in FIG. 11B, instrument interface 514 includes opening 522b through one side. Opening 522b may be shaped to correspond with hole 30a of retractor 30, as shown in FIGS. 4A-4D. As shown in FIG. 11A, opening 522b is aligned with threaded hole 522a on the opposite side of instrument interface 514. A reinforced or thickened portion 523 of instrument interface 514 may be provided surrounding threaded hole 522a to increase the strength of the adapter surrounding the hole and to provide sufficient thickness to accommodate internal threads.

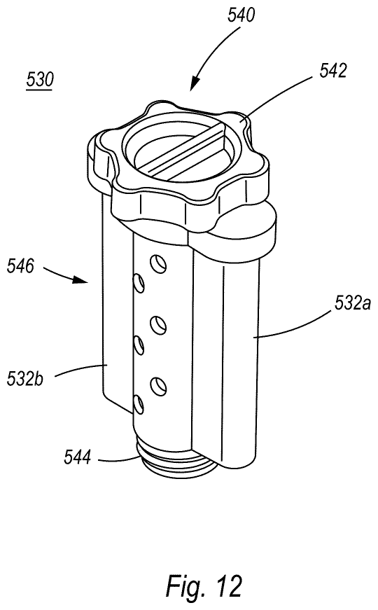

FIG. 12 shows key 530 for use with the adapter of FIGS. 11A and 11B. Key 530 is formed by key body 546. Key body 546 includes lobes 532a, 532b and has a cross section that allows the key to fit into opening 522b. Body 546 includes a cylindrical passage. Threaded member 540 is rotatably disposed through the passage. Threaded member 540 includes an end portion 544 that extends from body 546. Knob 542 is provided at an opposite end of threaded member 540.

In use, the handle of an instrument, such as retractor 30 is inserted into instrument interface 514 so that hole 30a aligns with opening 522b and threaded hole 522a. Key 540 is inserted into opening 522b and through hole 30a in the handle of the instrument. Lobes 532a, 532b engage with corresponding edges of opening 522b. According to one embodiment, lobes 532a, 532b are shaped to contact sides of hole 30a of instrument 30 to stabilize the connection between the instrument and adapter 510. Threaded member 540 engages with threaded hole 522a. Knob 542 is rotated to drive member 540 into the threaded hole. Tightening threaded member 540 secures key 530 with instrument interface 514 and secures the instrument with the adapter.

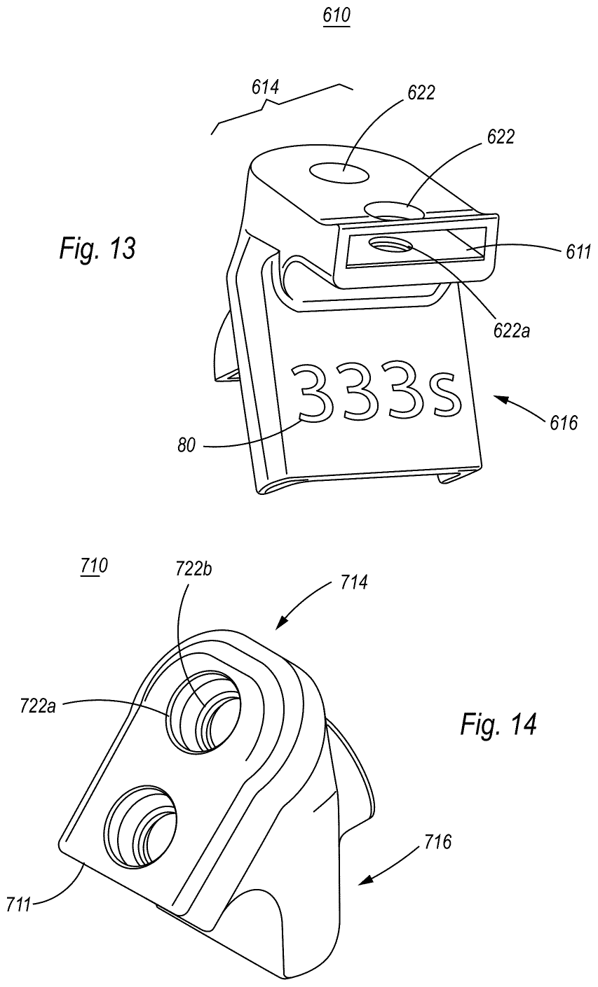

FIG. 13 shows adapter 610 according to another embodiment of the disclosure. Device interface 616 connects adapter 610 with device 12, as discussed with regard to previous embodiments. Instrument interface 614 has one or more circular openings 622. One or more threaded holes 622a are aligned with respective openings 622. According to one embodiment, adapter 610 includes indicia 80, such as lettering indented into or raised from the surface of the adapter. Indicia 80 may help practitioners to readily identify the configuration of the adapter, including the orientation of the device and instrument axes, during a surgical procedure. FIG. 14 shows another embodiment of adapter 710. Instrument interface 714 has two circular openings 722a that are aligned with threaded holes 722b.

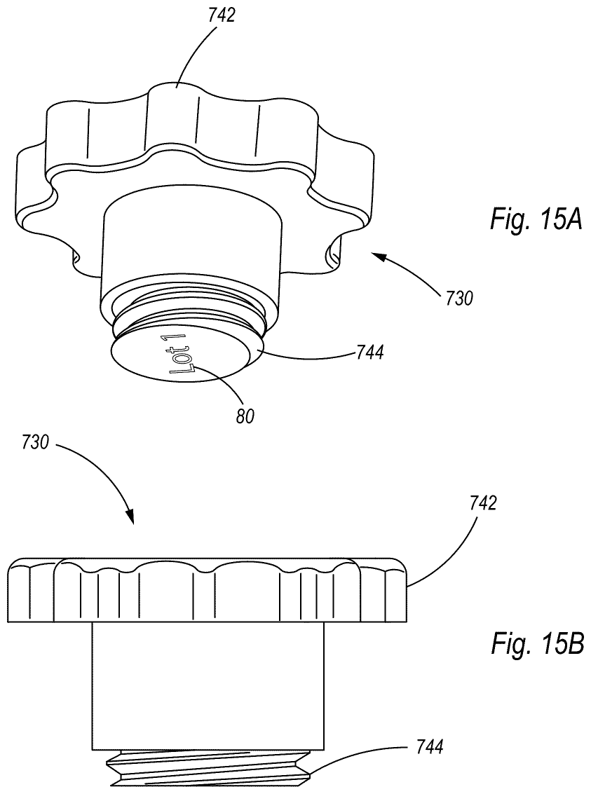

FIGS. 15A and 15B show a key 730 for securing instrument 30 with adapters 610 and 710 discussed above. Key 730 has a threaded end portion 744 and a knob 742. In use, the handle of an instrument is inserted into opening 611, 711 of adapter 610, 710. One or more keys 730 are inserted into openings 622, 722 so that end portion 744 contacts threaded hole 622a, 722a. Rotation of knob 742 engages threaded end 744 with threaded hole 622a,722a to secure the instrument with the adapter. Key 730 may include indicia 80.

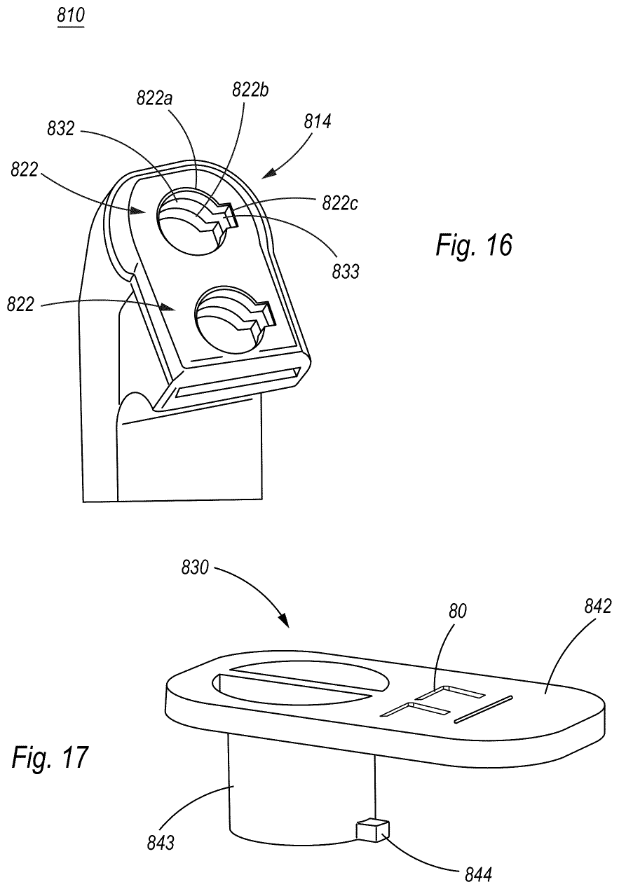

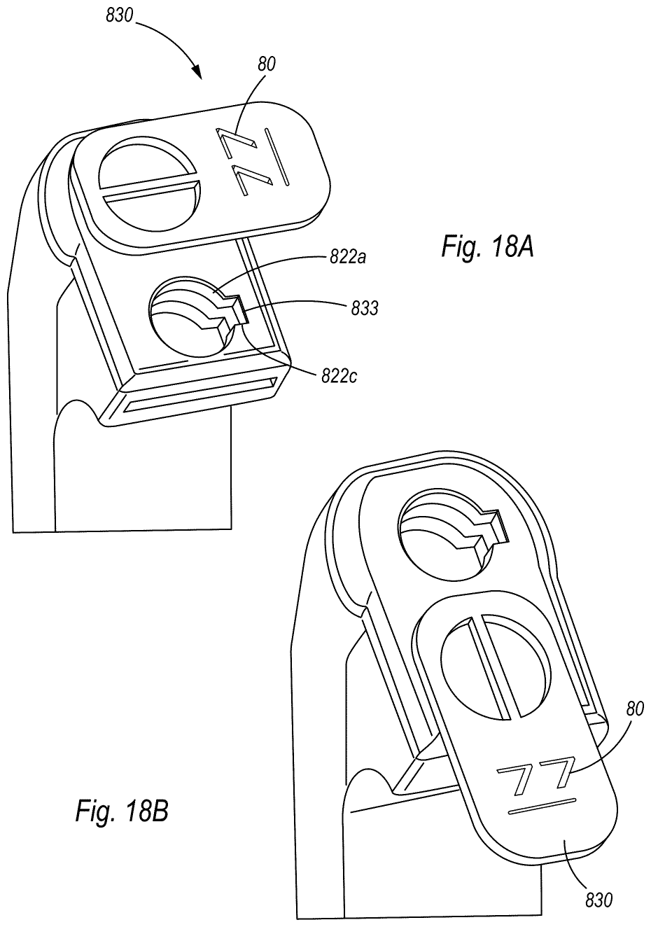

FIG. 16 shows an adapter 810 according to another embodiment of the disclosure. Instrument interface 814 includes two openings 822. Openings 822 have one or more key notches 830. Top portion 822a of opening 822 and bottom portion 822b of opening 822 are separated by a gap 832 that extends at least partially around the inner circumference of opening 822. FIG. 17 shows key 830 for use with adapter 810. Key 830 has a base plate 843 with a shaft 843 extending from one surface. Tooth 844 extends radially from shaft 843. According to one embodiment, shaft 843 engages holes 30a of instrument 30 so that by engaging key 830 with adapter 810, the instrument is secured with the adapter. Tooth 844 is shaped to fit through notch 833 of opening 822 and to slidably engage gap 832. When tooth 844 is aligned with notch 833, the key can be inserted into the opening. Tooth 844 is positioned along shaft 843 so that it aligns with gap 832. FIG. 18A shows key 840 with shaft 843 inserted into opening 822. Key 840 is then rotated, as shown in FIG. 18B so that the key is engaged with gap 832 to fix key with adapter 810 to secure the instrument with the adapter.

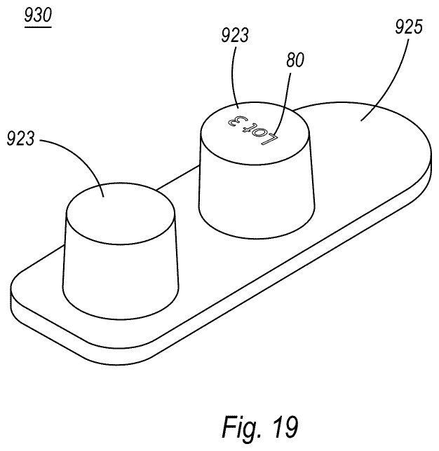

FIG. 19 shows key 930 for securing an instrument with adapters 610, 710, 820 discussed above. Key 930 may be used as an alternative to keys 730 and 830. Key 930 has a base plate 925 and one or more protrusions 923 extending from one surface of the plate. Protrusions 923 are sized and arranged on plate 925 so that the can be inserted into openings 622, 722, and 822 to engage with openings 30a of an instrument being connected with the adapter.

While illustrative embodiments of the disclosure have been described and illustrated above, it should be understood that these are exemplary of the disclosure and are not to be considered as limiting. Additions, deletions, substitutions, and other modifications can be made without departing from the spirit or scope of the disclosure. Accordingly, the disclosure is not to be considered as limited by the foregoing description.

We claim:

1. An adapter for connecting a surgical instrument with a self-retaining retraction device, the adapter comprising:
an instrument interface comprising cavity shaped to receive a proximal portion of the surgical instrument; and
a positioning device interface connected with the instrument interface and comprising a connection mechanism shaped to secure the adapter with the self-retaining retraction device.

2. The adapter of claim 1, further comprising a connecting portion connecting the instrument interface with the device interface, wherein the instrument interface defines an instrument axis, wherein the positioning device defines a device axis, and wherein the connecting portion positions the instrument axis and the device axis at a selected angular orientation with respect to one another in at least one geometric plane.

3. The adapter of claim 2, wherein the instrument axis and the device axis are oriented at selected angular orientations with respect to one another along two geometric planes.

4. The adapter of claim 2, wherein the selected angular orientation is between about 0 degrees and about 180 degrees.

5. The adapter of claim 4, wherein the selected angle is between about 45 degrees and about 135 degrees.

6. The adapter of claim 1, wherein instrument interface comprises a wall surrounding the cavity, wherein a keyhole extends through the wall on at least one side of the cavity, wherein a handle of the instrument comprises a hole positioned to align with the keyhole to form a keyway with the handle inserted into the cavity.

7. The adapter of claim 6, further comprising a key shaped to fit into the keyway.

8. The adapter of claim 7, wherein the key is secured with the keyway by an interference fit.

9. The adapter of claim 7, wherein the keyway comprise a plurality of keyways and wherein the key comprises a respective plurality of protrusions shaped to fit into respective ones of the keyways.

10. The adapter of claim 7, wherein the keyhole comprises a central region and one or more lobes extending from the central region.

11. The adapter of claim 7, wherein the instrument interface further comprises a threaded hole through the wall of the instrument interface on an opposite side of the cavity from the keyhole, wherein the threaded hole is aligned with the keyhole.

12. The adapter of claim 11, wherein the key comprises a threaded member and wherein the threaded member is engaged with the threaded hole to secure the key in the keyhole.

13. The adapter of claim 10, wherein the threaded member extends through the central region.

14. The adapter of claim 7, wherein the keyhole comprises a notch along a perimeter of the keyhole and a groove connected with the notch and extending at least partially along the circumference of an inner surface of the keyhole, wherein the key further comprises a tooth adapted fit within the notch and along the groove, wherein the tooth is moved along the notch and groove to engage the key with the keyhole.

15. The adapter of claim 1, wherein the self-retaining retraction device comprises an attachment mechanism comprising an opening at a distal end of the device and wherein the connection mechanism comprises a tongue shaped to fit through the opening and to engage with the attachment mechanism.

16. The adapter of claim 15, wherein the connection mechanism further comprises one or more walls positioned to engage outside surfaces of the device when the tongue is engaged with the attachment mechanism to secure the adapter with the device.

17. The adapter of claim 15, wherein the connection mechanism further comprises a lip extending from one or more of the walls and positioned to engage an outside top surface of the device.
